(19) United States
(12) Patent Application Publication
(10) Pub. No.: US 2026/0109931 A1
(43) Pub. Date: Apr. 23, 2026

(54) LONG-PERIOD HIGH-PRESSURE ENRICHMENT CULTURE AND ONLINE MONITORING APPARATUS AND METHOD FOR DEEP-SEA MICROORGANISMS

(71) Applicant: GUANGDONG LABORATORY OF SOUTHERN OCEAN SCIENCE AND ENGINEERING (GUANGZHOU), Guangdong (CN)

(72) Inventors: Jingchun FENG, Guangdong (CN); Si ZHANG, Guangdong (CN); Yongji HUANG, Guangdong (CN); Yingli ZHOU, Guangdong (CN); Zhifeng YANG, Guangdong (CN)

(73) Assignee: GUANGDONG LABORATORY OF SOUTHERN OCEAN SCIENCE AND ENGINEERING (GUANGZHOU), Guangdong (CN)

(21) Appl. No.: 19/365,224

(22) Filed: Oct. 22, 2025

(30) Foreign Application Priority Data
Oct. 22, 2024 (CN) 202411477984.2

(51) Int. Cl. C12M 1/34; C12M 1/00; C12M 1/26; C12M 1/36; C12M 3/06

(52) U.S. Cl. CPC C12M 41/40; C12M 23/16; C12M 23/58; C12M 29/06; C12M 33/14; C12M 41/12; C12M 41/26; C12M 41/34; C12M 41/36; C12M 41/48

(57) ABSTRACT

The present invention provides a long-period high-pressure enrichment culture and online monitoring apparatus and method for deep-sea microorganisms. The apparatus can simulate extreme deep-sea environments such as high pressure, low temperature, and oligotrophic conditions. Through pre-culture and graded serial culture, stable long-period growth and reproduction of the deep-sea microorganisms are ensured. An online system is used for monitoring the abundance, activity, and culture conditions of the microorganisms in real time to determine an appropriate timing for strain subculturing, and timely interactive subculturing and culture are performed, thereby achieving stable supply of high-abundance, high-activity microorganism enrichment culture solution. The present invention achieves precise pressure-retaining abundance counting of the microorganisms under in-situ pressure conditions and accurate control of a microbial growth cycle during enrichment culture, effectively improving timeliness and culture efficiency of the microorganism enrichment culture solution, and enabling long-period acquisition of high-abundance, high-activity microbial strain liquid.

### CROSS-REFERENCE TO RELATED APPLICATION

This application claims the priority benefit of China application serial no. 202411477984.2, filed on Oct. 22, 2024. The entirety of the above-mentioned patent application is hereby incorporated by reference herein and made a part of this specification.

### TECHNICAL FIELD

The present invention relates to the technical field of enrichment culture of marine microorganisms, and more specifically, to a long-period high-pressure enrichment culture and online monitoring apparatus and method for deep-sea microorganisms.

### BACKGROUND

The deep sea is characterized by complex geological landforms such as seamounts, ocean ridges, deep-sea plains, abyssal zones, and trenches, as well as unique chemoautotrophic ecosystems including hydrothermal vents and cold seeps. Due to the unique environment of high pressure, low temperature, darkness, and oligotrophic conditions, the deep sea harbors abundant resources of extremophilic microorganisms. The immense environmental pressure has driven deep-sea microorganisms to evolve specialized physiological structures and functions. These microorganisms can produce various bioactive substances with special physiological functions during their growth and metabolism to sustain their life activities. Therefore, the deep-sea microorganisms possess exceptionally high abundance and activity, as well as diversity in composition and function, forming unique species, gene types, and metabolic products through natural evolution, thereby having significant scientific and economic value. Development of new microbial resources from the deep sea, in-depth research into their biological characteristics, metabolic mechanisms, and ecological functions, and acquisition of unique bioactive substances or genetic resources represent the forefront of international research and development of new resources. However, due to the limitations of current conventional deep-sea microorganism sampling devices, microorganisms used for research often cannot maintain their original growth and metabolic activity. It is necessary to simulate in-situ extreme environments under laboratory conditions to activate and enrich microbial samples, thereby providing high-abundance, high-activity, and sufficient microbial strains for microbial resource exploration.

Due to the limitations of extreme environments, the growth and metabolic rates of most deep-sea microorganisms are extremely slow. During culture, the consumption of nutrients and the accumulation of intermediate metabolic wastes can inhibit microbial growth and reproduction. The most effective method is to transfer the microorganisms to a new culture environment. Microbial growth consists of a lag phase, a logarithmic phase, a stationary phase, and a decline phase. Determining the growth status of the microorganisms and selecting the enriched culture solution at the end of the logarithmic phase to the early stationary phase for subculturing is the key to obtaining high-activity, high-abundance strain suspensions. If an optimal subculturing timing is missed, the enriched culture solution enters the decline phase, resulting in massive microbial death, making the enriched culture solution unsuitable for subsequent microbial resource development, and causing the subcultured enriched culture solution to require a longer period to enter the logarithmic growth phase, thereby extending an enrichment culture cycle.

In the prior art, some deep-sea microorganism enrichment devices have been able to simulate the in-situ deep-sea environment under laboratory conditions for high-pressure enrichment culture of the microorganisms. For example, existing patent documents disclose a “rapid enrichment culture apparatus and method for marine anaerobic ammonium oxidation bacteria,” which enables the real-time detection of ammonia-nitrogen concentration of the culture solution in the reaction kettle to assess the growth and metabolic activity of anaerobic ammonium oxidation bacteria, and maintains the high ammonia-nitrogen metabolic capacity of the anaerobic ammonium oxidation bacteria by adjusting an injection volume of a low-salinity seawater tank. However, conventional monitoring methods, including this solution, can determine the subculturing timing only by monitoring environmental parameters such as carbon source, pH, electron acceptors, and metabolite concentrations. The monitoring of these environmental parameters cannot directly reflect the growth status of the microorganisms and has low accuracy. Currently, the most effective way to determine microbial growth is to perform cell counting on microorganisms in the enriched culture solution. A conventional counting method is cell abundance counting based on optical microscopy. However, this method is not suitable for cell counting of deep-sea high-pressure enriched culture solution, because a decompression process causes pressure shock to many deep-sea piezophilic bacteria, leading to their death and making it difficult to obtain accurate and valid microbial growth data under atmospheric pressure. Therefore, the development of high-pressure microbial abundance monitoring methods is key to the enrichment culture of the deep-sea microorganisms.

### SUMMARY

To overcome the shortcomings of the low abundance, activity, and timeliness of the enriched culture solution of deep-sea microorganism strains cultivated by the prior art, the present invention provides a long-period high-pressure enrichment culture and online monitoring apparatus and method for deep-sea microorganisms. The apparatus can simulate extreme deep-sea environments such as high pressure, low temperature, and oligotrophic conditions. Through pre-culture and graded serial culture, stable long-period growth and reproduction of the deep-sea microorganisms are ensured. An online system is used for monitoring the abundance, activity, and culture conditions of the microorganisms in real time to determine an appropriate timing for strain subculturing, and interactive subculturing and culture are performed, so the culture apparatus always maintains reserve microbial inoculum that meets the requirements for subsequent experiments, providing an important technical support for the development and utilization of deep-sea microbial resources.

To solve the above technical problems, the technical solutions of the present invention are as follows:

A long-period high-pressure enrichment culture and online monitoring apparatus for deep-sea microorganisms is provided and includes: a microorganism enrichment system, an online monitoring system, a temperature control system, a pressure control system, and a central control system; where
the microorganism enrichment system is provided with a plurality of reaction kettles, and is used for long-period enrichment culture and interactive subculturing of the deep-sea microorganisms;
the online monitoring system includes: an environmental parameter monitoring unit and a microbial abundance counting unit respectively connected to the microorganism enrichment system; the environmental parameter monitoring unit and the microbial abundance counting unit are respectively used for real-time monitoring of environmental parameters and biological abundance indicators of a culture solution in the microorganism enrichment system;
the temperature control system and the pressure control system are respectively connected to the microorganism enrichment system and are respectively used for regulating temperature and pressure within the microorganism enrichment system; the pressure control system is further connected to the microbial abundance counting unit and is used for achieving pressure-retaining counting on the microorganisms; and
a signal output end of the environmental parameter monitoring unit, a control end of the temperature control system, and a control end of the pressure control system are respectively connected to the central control system.

Preferably, the microorganism enrichment system includes: a plurality of reaction kettles that are connected in series and have an identical structure, each reaction kettle is of a detachable top-cover structure, and a sample transfer unit is provided between two of the reaction kettles connected in series;
a microbial culture solution is accommodated in each reaction kettle and a magnetic stir bar is provided in each reaction kettle; a bottom of each reaction kettle is provided with a magnetic stirrer, and the magnetic stirrer is used for driving the magnetic stir bar to rotate mechanically;
probes of the environmental parameter monitoring unit are respectively in contact with the culture solution inside each reaction kettle;
a sampling valve is provided outside each reaction kettle and is connected to the microfluidic chip in the microbial abundance counting unit; and
a top of each reaction kettle is provided with a gas injection valve and a vent valve, the gas injection valve is connected to the pressure control system, and each reaction kettle is connected to the temperature control system.

Preferably, the sample transfer unit includes: a regulating valve, a liquid injection pump, and a filter that are connected in sequence; and
the filter is specifically a stainless steel check valve, and the stainless steel check valve is internally provided with a stainless steel metal filtration membrane.

Preferably, the environmental parameter monitoring unit includes: a dissolved oxygen sensor, a pH sensor, and a Raman sensor;
probes of the dissolved oxygen sensor, pH sensor, and Raman sensor are respectively in contact with the culture solution inside each reaction kettle; and
signal output ends of the dissolved oxygen sensor, pH sensor, and Raman sensor are respectively electrically connected to the central control system.

Preferably, the microbial abundance counting unit includes: an observer, a microfluidic chip, and a fluorescence microscope;
both a top of the observer and a bottom of the observer are sapphire windows, such that excitation fluorescence of the fluorescence microscope passes through the microfluidic chip; the top of the observer is further provided with an intake valve and an exhaust valve, and the intake valve is connected to the pressure control system;
the microfluidic chip is fixed at a center of the observer by a clamping slot and is provided with a counting chamber, and a lower sample injection valve and an upper exhaust valve in communication with the counting chamber, the lower sample injection valve is connected to the sampling valve outside each reaction kettle and is used for injecting a microbial liquid from the reaction kettle into the counting chamber; the upper exhaust valve is used for evacuating gas in the counting chamber when the microbial liquid is injected into the counting chamber; and
a fluorescent dye for microbial staining is preloaded into the counting chamber; after the microbial liquid from the reaction kettle is mixed with the fluorescent dye for a specified period, fluorescent staining is completed; the observer is placed under the fluorescence microscope, and the fluorescence microscope is used for counting the abundance of the microorganisms.

Preferably, a bottom surface of the counting chamber is engraved with a grid for cell counting, and the grid comprises a plurality of squares with different sizes.

Preferably, the grid includes a large squares with a same size, each large square of the grid is further evenly divided into b medium squares, and each medium square is evenly divided into c small squares; where a, b, and c are a first positive integer, a second positive integer, and a third positive integer, respectively; and
the evenly divided large squares are used for cell counting and the concentration of the microorganisms in the microbial liquid is calculated based on the number of cells within the large squares.

Preferably, the temperature control system includes: a refrigeration unit, a refrigeration jacket, and a temperature sensor;
a control end of the refrigeration unit is connected to the central control system, an output end of the refrigeration unit is connected to the refrigeration jacket, and each reaction kettle is wrapped in the refrigeration jacket; the temperature sensor is provided inside each reaction kettle; and a signal output end of the temperature sensor is connected to the central control system.

Preferably, the pressure control system comprises: an air compressor, a booster pump, a gas storage tank, and a pressure regulating valve connected in sequence, as well as pressure sensors;
control ends of the air compressor, booster pump, and pressure regulating valve are connected to the central control system; an output end of the pressure regulating valve is connected to a gas injection valve of each reaction kettle and the intake valve of the observer; and
the pressure sensor is respectively provided inside each reaction kettle and inside the observer; and a signal output end of the pressure sensor is connected to the central control system.

The present invention further provides a long-period high-pressure enrichment culture and online monitoring method for deep-sea microorganisms based on the long-period high-pressure enrichment culture and online monitoring apparatus for deep-sea microorganisms; and the method includes the following steps:
S1: performing aseptic operations on all reaction kettles of a microorganism enrichment system and all components within an online monitoring system, and installing the long-period high-pressure enrichment culture and online monitoring apparatus for deep-sea microorganisms;
S2: sequentially adding deep-sea microorganism samples and activated culture media into each reaction kettle of the microorganism enrichment system, using a pressure control system and a temperature control system to regulate pressure and temperature within the microorganism enrichment system, and performing enrichment culture of the microorganisms under an environment with a preset pressure and a preset temperature;
S3: using an environmental parameter monitoring unit to monitor real-time changes in environmental parameters of the culture solution inside each reaction kettle; and when the environmental parameters meet preset conditions, injecting a microbial liquid from a corresponding reaction kettle into a microbial abundance counting unit;
S4: using the pressure control system to pressurize the microbial abundance counting unit, performing pressure-retaining counting on the microorganisms in the microbial abundance counting unit, and plotting a growth curve of microbial communities;
S5: determining an optimal subculturing timing for the microorganisms according to the growth curve, and at the optimal subculturing timing, transferring the microbial liquid from a previous-level reaction kettle to a next-level reaction kettle under pressure-retaining conditions; and
S6: repeating steps S3 to S5 to perform long-period enrichment culture and interactive subculturing of the deep-sea microorganism samples in the microorganism enrichment system.

Compared with the prior art, the beneficial effects of the technical solutions of the present invention are as follows:

The present invention provides a long-period high-pressure enrichment culture and online monitoring apparatus and method for deep-sea microorganisms. The apparatus can simulate extreme deep-sea environments such as high pressure, low temperature, and oligotrophic conditions. Through pre-culture and graded serial culture, stable long-period growth and reproduction of the deep-sea microorganisms are ensured. An online system is used for monitoring the abundance, activity, and culture conditions of the microorganisms in real time to determine an appropriate timing for strain subculturing, and interactive subculturing and culture are performed, thereby achieving stable supply of high-abundance, high-activity microorganism enrichment culture solution.

In the present invention, the optimal subculturing timing for enrichment culture can be determined by direct microorganism abundance counting combined with auxiliary environmental indicators. This addresses a difficulty in controlling the subculturing timing during enrichment culture of deep-sea microorganisms. Additionally, the present invention achieves the long-period acquisition of reserve microbial inoculum from the enrichment reaction kettle that meets the requirements for subsequent experiments, providing an important microbial strain support for the development and utilization of deep-sea microbial resources.

The present invention has broad applicability and can be used for enrichment culture of various deep-sea habitat microorganisms, without the need for specialized operational training. Pressure-retaining counting on microorganism abundance is similar to conventional blood cell counting, reducing labor and training costs. Compared with existing enrichment culture technologies, the present invention achieves precise control of a microbial growth cycle during enrichment, such that the microbial liquid in the enrichment culture reaction kettle always maintains a high growth rate and metabolic activity, ensuring the timeliness and culture efficiency of the microorganism enrichment culture solution.

### BRIEF DESCRIPTION OF DRAWINGS

FIG. 1 is an architecture diagram of a long-period high-pressure enrichment culture and online monitoring apparatus for deep-sea microorganisms provided in Embodiment 1.

FIG. 2 is a mechanical structure diagram of a long-period high-pressure enrichment culture and online monitoring apparatus for deep-sea microorganisms provided in Embodiment 2.

FIG. 3 is a schematic structural diagram of an observer provided in Embodiment 2.

FIG. 4 is a front view of a microfluidic chip provided in Embodiment 2.

FIG. 5 is a top view of the microfluidic chip provided in Embodiment 2 and a schematic structural diagram of a grid.

FIG. 6 is a schematic diagram showing a connection relationship between a central control system and other components provided in Embodiment 2.

FIG. 7 is a flowchart of long-period high-pressure enrichment culture and online monitoring for deep-sea microorganisms provided in Embodiment 3.

### DETAILED DESCRIPTION OF EMBODIMENTS

The drawings are for exemplary illustration only and shall not be construed as limitations to this patent.

To better illustrate this embodiment, certain components in the drawings may be omitted, enlarged, or reduced, and do not represent the dimensions of the actual product.

For those skilled in the art, it is understandable that certain well-known structures and descriptions thereof in the drawings may be omitted.

The technical solutions of the present invention are further illustrated below in conjunction with the drawings and embodiments.

### Embodiment 1

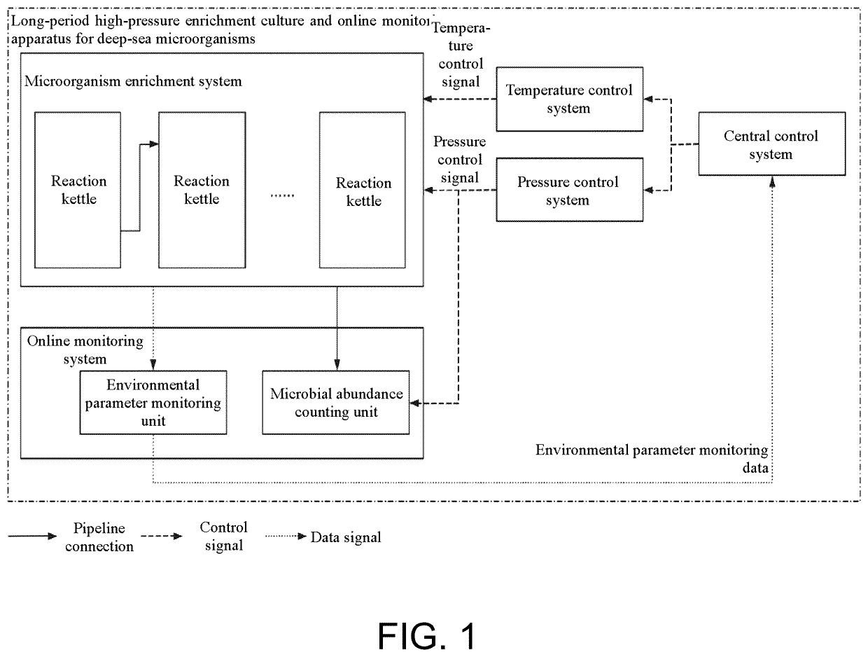

As shown in FIG. 1, this embodiment provides a long-period high-pressure enrichment culture and online monitoring apparatus for deep-sea microorganisms, and the apparatus includes: a microorganism enrichment system, an online monitoring system, a temperature control system, a pressure control system, and a central control system;
the microorganism enrichment system is provided with a plurality of reaction kettles, and is used for long-period enrichment culture and interactive subculturing of the deep-sea microorganisms;
the online monitoring system includes: an environmental parameter monitoring unit and a microbial abundance counting unit respectively connected to the microorganism enrichment system; the environmental parameter monitoring unit and the microbial abundance counting unit are respectively used for real-time monitoring of environmental parameters and biological abundance indicators of a culture solution in the microorganism enrichment system;
the temperature control system and the pressure control system are respectively connected to the microorganism enrichment system and are respectively used for regulating temperature and pressure within the microorganism enrichment system; the pressure control system is further connected to the microbial abundance counting unit and is used for achieving pressure-retaining counting on the microorganisms; and
a signal output end of the environmental parameter monitoring unit, a control end of the temperature control system, and a control end of the pressure control system are respectively connected to the central control system.

In a specific implementation process, aseptic operations are performed on all reaction kettles of a microorganism enrichment system and all components within an online monitoring system, and the long-period high-pressure enrichment culture and online monitoring apparatus for deep-sea microorganisms is installed.

Deep-sea microorganism samples and activated culture media are sequentially added into each reaction kettle of the microorganism enrichment system; a pressure control system and a temperature control system are used to regulate pressure and temperature within the microorganism enrichment system, and enrichment culture is performed on the microorganisms under an environment with a preset pressure and a preset temperature.

An environmental parameter monitoring unit is used to monitor real-time changes in environmental parameters of the culture solution inside each reaction kettle; and when the environmental parameters meet preset conditions, a microbial liquid from a corresponding reaction kettle is injected into a microfluidic chip of a microbial abundance counting unit.

The pressure control system is used to pressurize the microbial abundance counting unit; pressure-retaining counting is performed on the microorganisms under the fluorescence microscope; and a growth curve of microbial communities is plotted.

An optimal subculturing timing for the microorganisms is determined according to the growth curve, and at the optimal subculturing timing, the microbial liquid is transferred from a previous-level reaction kettle to a next-level reaction kettle under pressure-retaining conditions.

The above steps are repeated to perform long-period enrichment culture and interactive subculturing of the deep-sea microorganism samples in the microorganism enrichment system.

The apparatus can perform precise pressure-retaining abundance counting on the microorganisms under in-situ pressure conditions, such that a microbial growth cycle is accurately controlled during enrichment culture. This addresses a difficulty in controlling the subculturing timing during enrichment culture, effectively improving timeliness and culture efficiency of the microorganism enrichment culture solution, and enabling long-period acquisition of high-abundance, high-activity microbial strain liquid, thus providing a solid source of basic strains for subsequent development of deep-sea microorganism resources.

### Embodiment 2

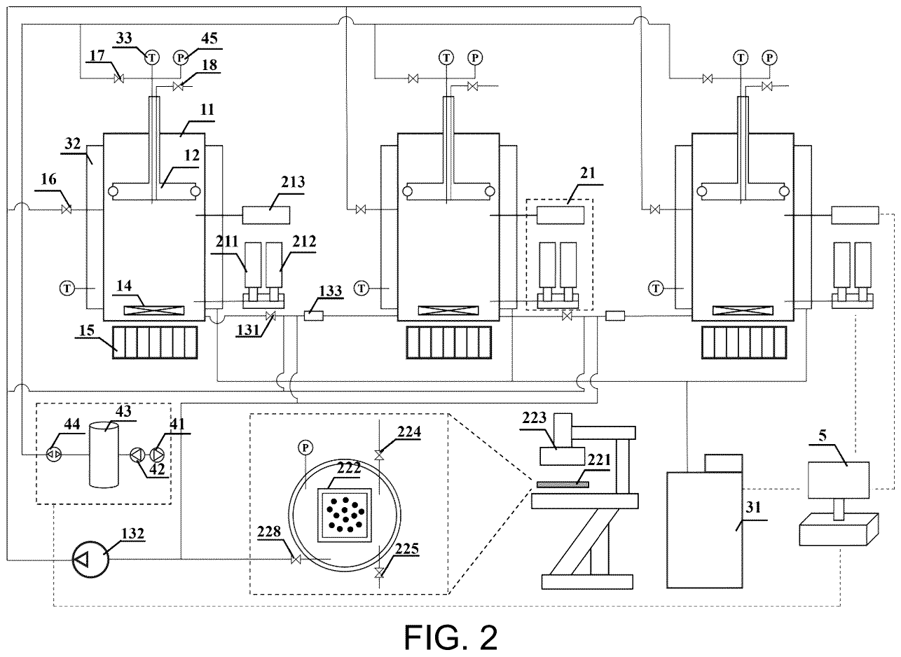

As shown in FIG. 2, this embodiment provides a long-period high-pressure enrichment culture and online monitoring apparatus for deep-sea microorganisms, and the apparatus includes: a microorganism enrichment system 1, an online monitoring system 2, a temperature control system 3, a pressure control system 4, and a central control system 5.

The microorganism enrichment system 1 is provided with a plurality of reaction kettles 11, and is used for long-period enrichment culture and interactive subculturing of the deep-sea microorganisms.

The online monitoring system 2 includes: an environmental parameter monitoring unit 21 and a microbial abundance counting unit 22 respectively connected to the microorganism enrichment system 1; the environmental parameter monitoring unit 21 and the microbial abundance counting unit 22 are respectively used for real-time monitoring of environmental parameters and biological abundance indicators of a culture solution in the microorganism enrichment system 1.

The microbial abundance counting unit 22 is provided with a microfluidic chip 222 and a fluorescence microscope 223; a fluorescent dye for microbial staining is preloaded into the microfluidic chip 222, and after fluorescence staining is performed on the microbial liquid from the microorganism enrichment system 1, microorganism abundance counting is performed using the fluorescence microscope 223.

The temperature control system 3 and the pressure control system 4 are respectively connected to the microorganism enrichment system 1 and are respectively used for regulating temperature and pressure within the microorganism enrichment system 1; the pressure control system 4 is further connected to the microbial abundance counting unit 22 and is used for achieving pressure-retaining counting on the microorganisms.

A signal output end of the environmental parameter monitoring unit 21, a control end of the temperature control system 3, and a control end of the pressure control system 4 are respectively connected to the central control system 5.

The microorganism enrichment system 1 includes: a plurality of reaction kettles 11 that are connected in series and have an identical structure, and each reaction kettle 11 is of a high-pressure-resistant detachable top-cover structure 12, which facilitates placement of nutrient substrates and simplifies operation.

A sample transfer unit 13 is provided between two of the reaction kettles 11 connected in series and used for interactive subculturing of microbial liquid between the reaction kettles 11 during the later culture phase; and in this embodiment, the sample transfer unit 13 includes: a regulating valve 131, a liquid injection pump 132, and a filter 133 connected in sequence.

The filter 133 is specifically a stainless steel check valve, and the stainless steel check valve is internally provided with a 10 μm stainless steel metal filtration membrane. This effectively intercepts particulate sediments and certain particulate metabolites from original samples without affecting the passage of microorganisms, thereby improving the inoculation efficiency of the microorganisms during the subculturing process.

To increase the utilization efficiency of nutrients in the microorganisms during culture, a microbial culture solution is accommodated in each reaction kettle 11 and a magnetic stir bar 14 is provided in each reaction kettle; a bottom of each reaction kettle 11 is provided with a magnetic stirrer 15 and the magnetic stirrer 15 is used for driving the magnetic stir bar 14 to rotate mechanically, enhancing mass transfer between microorganisms and nutrient solutions. This addresses a difficulty that manual stirring rods can only perform indirect stirring and are unfavorable for reaction kettle sealing, thereby achieving real-time homogenization of the enrichment culture solution during culture.

Probes of the environmental parameter monitoring unit 21 are respectively in contact with the culture solution inside each reaction kettle 11;
the sampling valve 16 is provided outside each reaction kettle 11 and is connected to the microfluidic chip 222 in the microbial abundance counting unit 22; and the sampling valve 16 is used for collecting the microbial liquid from the reaction kettle 11 and subsequent pressure-retaining biological abundance counting.

A top of each reaction kettle 11 is provided with a gas injection valve 17 and a vent valve 18, the gas injection valve 17 is connected to the pressure control system 4 and is used for controlling pressurization and depressurization of a culture system, such that pressure inside the reaction kettle 11 reaches in-situ environmental pressure of a culture sample.

Each reaction kettle 11 is connected to the temperature control system 3, and the temperature in the reaction kettle 11 is regulated by the temperature control system 3 to be consistent with in-situ temperature of the deep-sea environment.

The environmental parameter monitoring unit 21 includes: a dissolved oxygen sensor 211, a pH sensor 212, and a Raman sensor 213.

Probes of the dissolved oxygen sensor 211, pH sensor 212, and Raman sensor 213 are respectively in contact with the culture solution in each reaction kettle 11, and can be used for monitoring changes in dissolved oxygen, pH value, and organic nutrient concentration in the enrichment culture solution.

Signal output ends of the dissolved oxygen sensor 211, pH sensor 212, and Raman sensor 213 are respectively electrically connected to the central control system 5, and the specific values are directly displayed on the central control system 5.

Through monitoring of the above indicators, the culture process can be ensured to be always under anaerobic conditions, and the consumption of organic matter and the growth of acidic metabolic waste in the culture can be reflected. These indicators can serve as indirect signals for subculture inoculation.

The microbial abundance counting unit 22 includes: a high-pressure-resistant stainless steel observer 221, a microfluidic chip 222, and a fluorescence microscope 223.

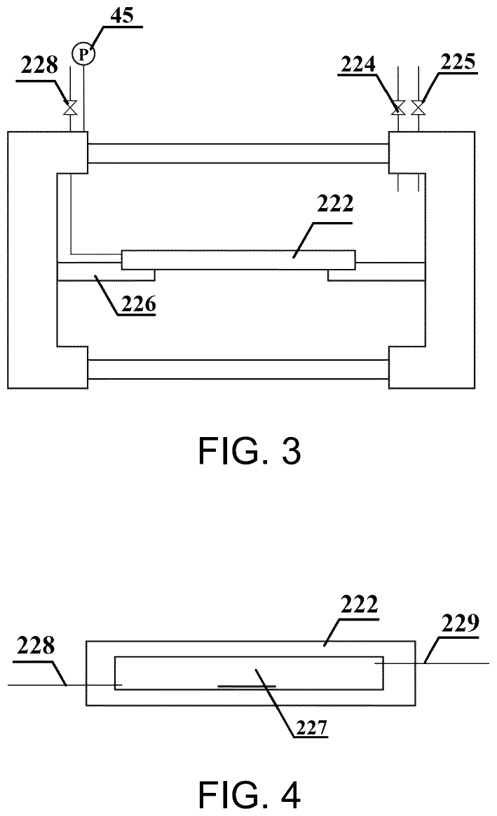

As shown in FIG. 3, a top of the observer 221 and of the observers are sapphire windows, such that excitation fluorescence of the fluorescence microscope 223 passes through the microfluidic chip 222; the observer 221 should not be too thick, with a thickness preferably less than 5 cm, to prevent the fluorescence microscope 223 from failing to focus on the microfluidic chip 222.

To enable the microorganism counting process to take place under high-pressure conditions, the top of the observer 221 is further provided with an intake valve 224 and an exhaust valve 225. The intake valve 224 is connected to the pressure control system 4. During counting, the intake valve 224 is used for pressurization until the pressure inside the observer 221 is consistent with the pressure inside the reaction kettle 11. After counting, the exhaust valve 225 is used to depressurize the observer 221. The entire counting process is performed under pressure, preventing the death of piezophilic bacteria due to pressure loss and ensuring the reliability of microorganism counting.

The microfluidic chip 222 is fixed at the center of the observer 221 by a clamping slot 226 to prevent disturbance caused by gas injection and liquid injection.

As shown in FIG. 4, the microfluidic chip 222 is provided with a counting chamber 227, and the lower sample injection valve and the upper exhaust valve in communication with the counting chamber 227, the lower sample injection valve 228 is connected to a sampling valve 16 outside each reaction kettle 11 and is used for injecting a microbial liquid from the reaction kettle 11 into the counting chamber 227; the upper exhaust valve 229 is used for evacuating gas in the counting chamber 227 when the microbial liquid is injected into the counting chamber 227, such that the enriched culture solution can be successfully and stably injected.

A fluorescent dye for microbial staining is preloaded into the counting chamber 227; after the microbial liquid from the reaction kettle 11 is mixed with the fluorescent dye for a specified period, fluorescent staining is completed; the observer 221 is placed under the fluorescence microscope 223, and the fluorescence microscope 223 is used for counting the abundance of the microorganisms.

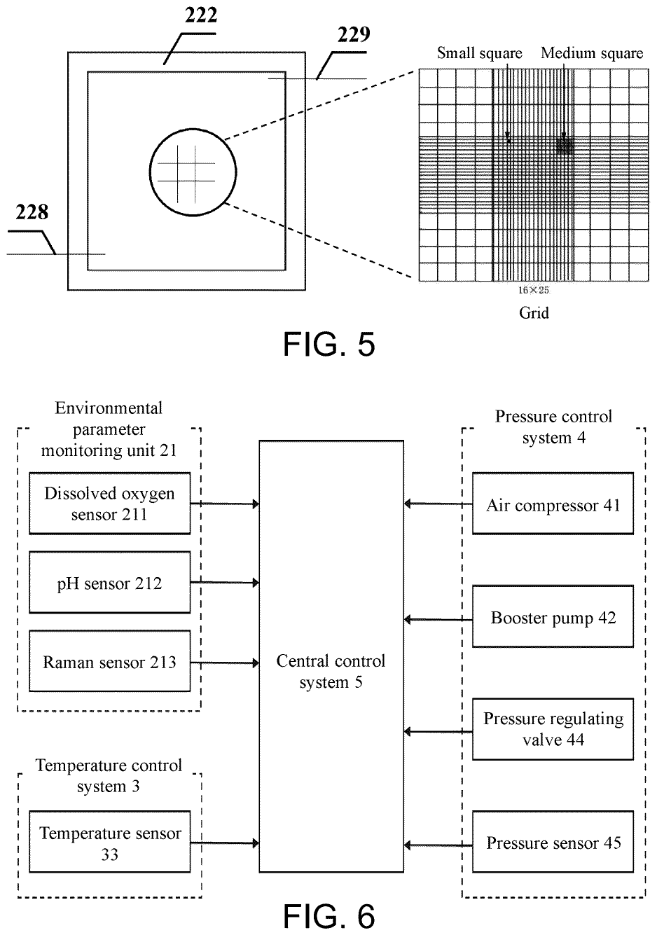

As shown in FIG. 5, a bottom surface of the counting chamber 227 is engraved with a grid for cell counting, and the grid includes a plurality of squares with different sizes.

In this embodiment, the counting chamber 227 has a thickness of 0.1 mm, a bottom surface size of 2 cm×2 cm, and a volume of 40 μL, and can accommodate 40 μL of staining solution and microbial liquid.

The grid includes nine large squares with the same size, each large square having a side length of 1 mm and a volume of 0.1 mm³.

The central large square of the grid is further evenly divided into sixteen medium squares, and each medium square is evenly divided into twenty-five small squares.

The central large square of the grid is used for cell counting. According to the total number of cells in the central large square and the volume of the large square, the concentration of microorganisms in the microbial liquid is calculated.

The temperature control system 3 includes: a refrigeration unit 31, a refrigeration jacket 32, and a temperature sensor 33.

A control end of the refrigeration unit 31 is connected to the central control system 5, an output end of the refrigeration unit 31 is connected to the refrigeration jacket 32, and each reaction kettle 11 is wrapped in the refrigeration jacket 32; the refrigeration jacket 32 in this embodiment is specifically a water bath jacket; water bath refrigeration allows for more precise and uniform temperature control; the temperature sensor 33 is provided inside each reaction kettle 11; and a signal output end of the temperature sensor 33 is connected to the central control system 5.

The pressure control system 4 includes: an air compressor 41, a booster pump 42, a gas storage tank 43, and a pressure regulating valve 44 connected in sequence, as well as a pressure sensor 45.

Control ends of the air compressor 41, booster pump 42, and pressure regulating valve 44 are connected to the central control system 5; an output end of the pressure regulating valve 44 is connected to a gas injection valve 17 of each reaction kettle 11 and the intake valve 224 of the observer 221.

The pressure sensor 45 is respectively provided inside each reaction kettle 11 and inside the observer 221; and a signal output end of the pressure sensor 45 is connected to the central control system 5.

In this embodiment, as shown in FIG. 6, the central control system 5 is electrically connected to the control ends of the air compressor 41, booster pump 42, and pressure regulating valve 44 of the pressure control system 4, the signal output end of the pressure sensor 45, the control end of the refrigeration system 31 of the temperature control system 3, the signal output end of the temperature sensor 33 of the temperature control system 3, and the signal output ends of various sensors of the environmental parameter monitoring unit 21, and is used for r temperature and pressure regulation of the reaction kettle 11 and real-time data acquisition, processing, and storage of environmental parameter changes of the enriched culture solution.

In a specific implementation process, aseptic operations are performed on all reaction kettles 11 of a microorganism enrichment system 1 and all components within an online monitoring system 2, and the long-period high-pressure enrichment culture and online monitoring apparatus for deep-sea microorganisms is installed.

Deep-sea microorganism samples and activated culture media are sequentially added into each reaction kettle 11 of the microorganism enrichment system 1; a pressure control system 4 and a temperature control system 3 are used to regulate pressure and temperature within the microorganism enrichment system 1, and enrichment culture is performed on the microorganisms under high-pressure and low-temperature conditions.

An environmental parameter monitoring unit 21 is used to monitor real-time changes in environmental parameters of the culture solution inside each reaction kettle 11; and when the environmental parameters meet preset conditions, a microbial liquid from a corresponding reaction kettle 11 is injected into a microfluidic chip 222 of a microbial abundance counting unit 22.

The pressure control system 4 is used to pressurize the observer 221 of the microbial abundance counting unit 22; pressure-retaining counting is performed on the microorganisms under the fluorescence microscope 223; and a growth curve of microbial communities is plotted.

An optimal subculturing timing for the microorganisms is determined according to the growth curve, and at the optimal subculturing timing, the microbial liquid is transferred from a previous-level reaction kettle 11 to a next-level reaction kettle 11 under pressure-retaining conditions.

The above steps are repeated to perform long-period enrichment culture and interactive subculturing of the deep-sea microorganism samples in the microorganism enrichment system 1.

The apparatus can perform precise pressure-retaining abundance counting on the microorganisms under in-situ pressure conditions, such that a microbial growth cycle is accurately controlled during enrichment culture. This addresses a difficulty in controlling the subculturing timing during enrichment culture, effectively improving timeliness and culture efficiency of the microorganism enrichment culture solution, and enabling long-period acquisition of high-abundance, high-activity microbial strain liquid, thus providing a solid source of basic strains for subsequent development of deep-sea microorganism resources.

### Embodiment 3

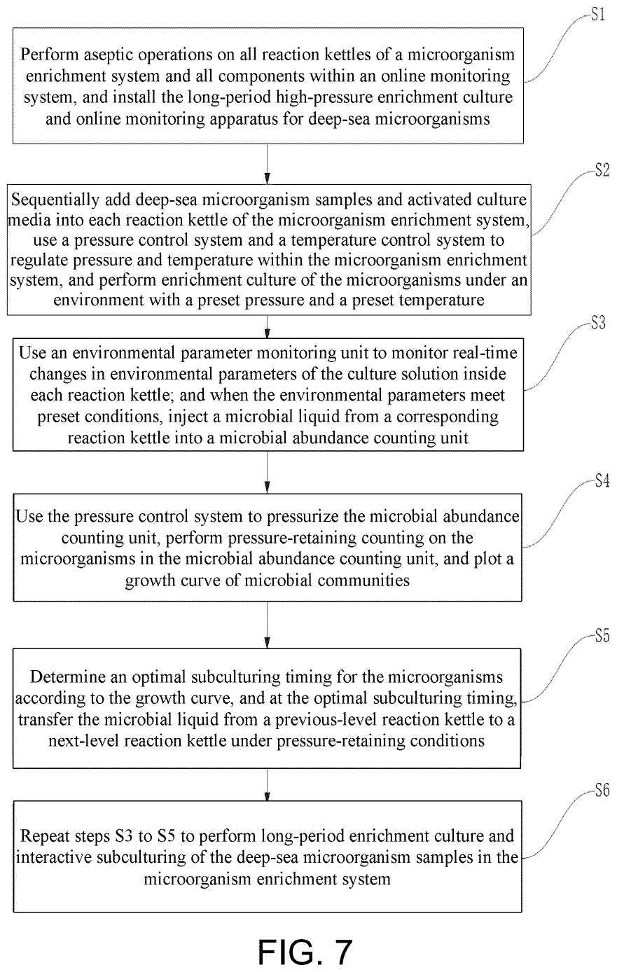

As shown in FIG. 7, this embodiment provides a long-period high-pressure enrichment culture and online monitoring method for deep-sea microorganisms based on the long-period high-pressure enrichment culture and online monitoring apparatus for deep-sea microorganisms in Embodiment 2; and the method includes the following steps:
S1: performing aseptic operations on all reaction kettles of a microorganism enrichment system and all components within an online monitoring system, and installing the long-period high-pressure enrichment culture and online monitoring apparatus for deep-sea microorganisms;
S2: sequentially adding deep-sea microorganism samples and activated culture media into each reaction kettle of the microorganism enrichment system, using a pressure control system and a temperature control system to regulate pressure and temperature within the microorganism enrichment system, and performing enrichment culture of the microorganisms under an environment with a preset pressure and a preset temperature;
S3: using an environmental parameter monitoring unit to monitor real-time changes in environmental parameters of the culture solution inside each reaction kettle; and when the environmental parameters meet preset conditions, injecting a microbial liquid from a corresponding reaction kettle into a microbial abundance counting unit;
S4: using the pressure control system to pressurize the microbial abundance counting unit, performing pressure-retaining counting on the microorganisms in the microbial abundance counting unit, and plotting a growth curve of microbial communities;
S5: determining an optimal subculturing timing for the microorganisms according to the growth curve, and at the optimal subculturing timing, transferring the microbial liquid from a previous-level reaction kettle to a next-level reaction kettle under pressure-retaining conditions; and
S6: repeating steps S3 to S5 to perform long-period enrichment culture and interactive subculturing of the deep-sea microorganism samples in the microorganism enrichment system.

In a specific implementation process, Step 1: before culture, aseptic operations are performed on the reaction kettle 11 and its associated pipe and valve components by exposure to 75% ethanol or ultraviolet light, and then the reaction kettle 11 and its associated components are transferred to a clean bench for being assembled in aseptic conditions. Generally, before a specific microbial taxon is selected for enrichment, the microbial community in the deep-sea sample is activated to allow most microbial taxa in the sample to resume functional metabolism. The activated microbial liquid is then inoculated under pressure into a new enrichment culture reaction kettle for enrichment culture of specific microbial taxa. Therefore, in the aseptic conditions, deep-sea microorganism samples (such as deep-sea sediments or seawater), microbial activation culture media (depending on the sample and the desired microbial taxa), and a magnetic stir bar 14 are sequentially added to the reaction kettle 11, and then various valves and sensor components are installed.

Step 2: A gas injection valve 17 of the reaction kettle 11 is connected to a pressure regulating valve 44 of a pressure control system 4; an air compressor 41, a booster pump 42, and a pressure regulating valve 44 are sequentially regulated; and the gas injection valve 17 is opened such that a pressure value of a pressure sensor 45 in the reaction kettle 11 reaches a preset value. Then, a refrigeration jacket 32 of a temperature control system 3 is wrapped outside the reaction kettle 11, and a refrigeration system 31 is started such that a temperature value of a temperature sensor 33 in the reaction kettle 11 reaches a preset value. Finally, a magnetic stirrer 15 is started to drive the magnetic stir bar 14 therein for stirring. The entire activation culture process is performed under high-pressure and low-temperature conditions.

Step 3: During culture, a dissolved oxygen sensor 211, a pH sensor 212, and a Raman sensor 213 are used for in real time monitoring changes in dissolved oxygen, pH value, and organic nutrient concentration in the enrichment culture solution. Values obtained through monitoring are displayed in real time on the central control system 5, indirectly reflecting the growth status of the microorganisms. When the pH value of the culture solution drops by 2 to 3 orders of magnitude and the concentrations of organic nutrients such as glucose and acetate decrease significantly, this indicates that nutrients in the culture solution have been largely consumed and acidic metabolites have been generated, and subculturing may be considered.

Step 4: Every 1 to 2 days, cell counting is performed on the microbial liquid in the culture solution. Specifically, 20 μL of DAPI fluorescent dye is injected into a microfluidic chip 222, and then the microfluidic chip 222 is placed in an observer 221 and fixed with a clamping slot 226. An intake valve 224 of the observer 221 is connected to the pressure regulating valve 44 of the pressure control system 4, and the air compressor 41, the booster pump 42, the pressure regulating valve 44, and the intake valve 224 are sequentially controlled such that the internal pressure of the observer 221 is increased to be consistent with the internal pressure of the reaction kettle 11. Then, a lower sample injection valve 228 of the observer 221 is sequentially connected to a liquid injection pump 132 and a sampling valve 16 of the reaction kettle 11. The liquid injection pump 132 is started to slowly and quantitatively inject 20 μL of the culture solution into the microfluidic chip 222. The sample is incubated at low temperature for 15-20 minutes to allow fluorescent labeling of the microorganisms. After staining, the microfluidic chip 222 is placed under a fluorescence microscope 223. A focal length of the fluorescence microscope 223 is regulated to find grids in a counting chamber 227 under a low-power microscope, then switched to a high-power microscope to count the total number of cells in the central large grid within the field of view, and the total number of cells is then converted to the bacterial concentration of the inoculum. The results of cell counting are used to plot the growth curve of the microbial communities, so as to determine the lag phase, logarithmic phase, stationary phase, and decline phase of the microbial communities.

Step 5: An optimal subculturing timing is determined according to the growth curve and the changes in environmental parameters, and generally from the end of the logarithmic growth phase to the early stationary phase. Before subculturing, steps 1 and 2 are repeated, but the activation culture solution is replaced with the enriched culture solution specific to the desired microbial taxa. During subculturing, the bottom of the previous-level reaction kettle 11 is sequentially connected to the regulating valve 131, the liquid injection pump 132, and the filter 133 via pipelines, and then the filter 133 is connected to the regulating valve 131 of the next-level reaction kettle 11. The regulating valves 131 at two ends are opened to connect the two reaction kettles 11, and the enriched culture solution from the previous enrichment reaction kettle 11 is quantitatively inoculated into the next-level reaction kettle 11 under pressure through the liquid injection pump 132.

Step 6: Steps 3 to 6 are repeated to perform long-period enrichment culture and interactive subculturing of the microbial liquid in the reaction kettle 11.

Through the above steps, the microbial culture solution from the next-level reaction kettle 11 can maintain a high growth rate, and high-abundance, and the high-activity microbial enriched culture solution can be obtained from the previous-level reaction kettle 11. This provides a sufficient source of microbial strains for the development and utilization of deep-sea microorganism resources over a long period of time.

Identical or similar reference numerals correspond to identical or similar components.

Terms describing positional relationships in the drawings are for exemplary illustration only and shall not be construed as limitations to this patent.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

What is claimed is:

1. A long-period high-pressure enrichment culture and online monitoring apparatus for deep-sea microorganisms, comprising: a microorganism enrichment system, an online monitoring system, a temperature control system, a pressure control system, and a central control system;
wherein
the microorganism enrichment system is provided with a plurality of reaction kettles, and is used for long-period enrichment culture and interactive subculturing of the deep-sea microorganisms;
the online monitoring system includes: an environmental parameter monitoring unit and a microbial abundance counting unit respectively connected to the microorganism enrichment system; the environmental parameter monitoring unit and the microbial abundance counting unit are respectively used for real-time monitoring of environmental parameters and biological abundance indicators of a culture solution in the microorganism enrichment system;
the microbial abundance counting unit comprises: an observer, a microfluidic chip, and a fluorescence microscope;
a top of the observer and a bottom of the observer are sapphire windows, such that excitation fluorescence of the fluorescence microscope passes through the microfluidic chip; the top of the observer is further provided with an intake valve and an exhaust valve, and the intake valve is connected to the pressure control system;
the microfluidic chip is fixed at a center of the observer by a clamping slot and is provided with a counting chamber, and a lower sample injection valve and an upper exhaust valve in communication with the counting chamber, the lower sample injection valve is connected to a sampling valve outside each of the reaction kettles and is used for injecting a microbial liquid from the reaction kettle into the counting chamber; the upper exhaust valve is used for evacuating gas in the counting chamber when the microbial liquid is injected into the counting chamber; and
a fluorescent dye for microbial staining is preloaded into the counting chamber; after the microbial liquid from the reaction kettle is mixed with the fluorescent dye for a specified period, fluorescent staining is completed; the observer is placed on the fluorescence microscope, and the fluorescence microscope is used for counting the abundance of the microorganisms;
the temperature control system and the pressure control system are respectively connected to the microorganism enrichment system and are respectively used for regulating temperature and pressure within the microorganism enrichment system; the pressure control system is further connected to the microbial abundance counting unit and is used for achieving pressure-retaining counting on the microorganisms; and
a signal output end of the environmental parameter monitoring unit, a control end of the temperature control system, and a control end of the pressure control system are respectively connected to the central control system.

2. The long-period high-pressure enrichment culture and online monitoring apparatus for deep-sea microorganisms according to claim 1, wherein the microorganism enrichment system comprises: the plurality of reaction kettles that are connected in series and have an identical structure, each of the reaction kettles is of a detachable top-cover structure, and a sample transfer unit is provided between two of the reaction kettles connected in series;
a microbial culture solution is accommodated in each of the reaction kettles and a magnetic stir bar is provided in each of the reaction kettles; a bottom of each of the reaction kettles is provided with a magnetic stirrer, and the magnetic stirrer is used for driving the magnetic stir bar to rotate mechanically;
probes of the environmental parameter monitoring unit are respectively in contact with the culture solution inside each of the reaction kettles;
the sampling valve is provided outside each of the reaction kettles and is connected to the microfluidic chip in the microbial abundance counting unit; and
a top of each of the reaction kettles is provided with a gas injection valve and a vent valve, the gas injection valve is connected to the pressure control system, and each of the reaction kettles is connected to the temperature control system.

3. The long-period high-pressure enrichment culture and online monitoring apparatus for deep-sea microorganisms according to claim 2, wherein the sample transfer unit comprises: a regulating valve, a liquid injection pump, and a filter that are connected in sequence; and
the filter is specifically a stainless steel check valve, and the stainless steel check valve is internally provided with a stainless steel metal filtration membrane.

4. The long-period high-pressure enrichment culture and online monitoring apparatus for deep-sea microorganisms according to claim 2, wherein the environmental parameter monitoring unit comprises: a dissolved oxygen sensor, a pH sensor, and a Raman sensor;
probes of the dissolved oxygen sensor, the pH sensor, and the Raman sensor are respectively in contact with the culture solution inside each of the reaction kettles; and
signal output ends of the dissolved oxygen sensor, pH sensor, and Raman sensor are respectively electrically connected to the central control system.

5. The long-period high-pressure enrichment culture and online monitoring apparatus for deep-sea microorganisms according to claim 1, wherein a bottom surface of the counting chamber is engraved with a grid for cell counting, and the grid comprises a plurality of squares with different sizes.

6. The long-period high-pressure enrichment culture and online monitoring apparatus for deep-sea microorganisms according to claim 5, wherein the grid comprises a large squares with a same size, any one of the large square of the grid is further evenly divided into b medium squares, and each of the medium squares is evenly divided into c small squares; wherein a, b, and c are a first positive integer, a second positive integer, and a third positive integer, respectively; and
the evenly divided large squares are used for cell counting and the concentration of the microorganisms in the microbial liquid is calculated based on a number of cells within the large squares.

7. The long-period high-pressure enrichment culture and online monitoring apparatus for deep-sea microorganisms according to claim 2, wherein the temperature control system comprises: a refrigeration unit, a refrigeration jacket, and a temperature sensor;
a control end of the refrigeration unit is connected to the central control system, an output end of the refrigeration unit is connected to the refrigeration jacket, and each of the reaction kettles is wrapped in the refrigeration jacket; the temperature sensor is provided inside each of the reaction kettles; and a signal output end of the temperature sensor is connected to the central control system.

8. The long-period high-pressure enrichment culture and online monitoring apparatus for deep-sea microorganisms according to claim 1, wherein the pressure control system comprises: an air compressor, a booster pump, a gas storage tank, and a pressure regulating valve connected in sequence, as well as pressure sensors;
control ends of the air compressor, booster pump, and pressure regulating valve are connected to the central control system; an output end of the pressure regulating valve is connected to a gas injection valve of each of the reaction kettles and the intake valve of the observer; and
the pressure sensor is respectively provided inside each of the reaction kettles and inside the observer; and a signal output end of the pressure sensor is connected to the central control system.

9. A long-period high-pressure enrichment culture and online monitoring method for deep-sea microorganisms based on the long-period high-pressure enrichment culture and online monitoring apparatus for deep-sea microorganisms according to claim 1, comprising the following steps:
S1: performing aseptic operations on all of the reaction kettles of the microorganism enrichment system and all components within the online monitoring system, and installing the long-period high-pressure enrichment culture and online monitoring apparatus for deep-sea microorganisms;
S2: sequentially adding deep-sea microorganism samples and activated culture media into each of the reaction kettles of the microorganism enrichment system, using the pressure control system and the temperature control system to regulate pressure and temperature within the microorganism enrichment system, and performing enrichment culture of the microorganisms under an environment with a preset pressure and a preset temperature;
S3: using an environmental parameter monitoring unit to monitor real-time changes in environmental parameters of the culture solution inside each of the reaction kettles; and when the environmental parameters meet preset conditions, injecting the microbial liquid from a corresponding reaction kettle into the microbial abundance counting unit;
S4: using the pressure control system to pressurize the microbial abundance counting unit, performing pressure-retaining counting on the microorganisms, and plotting a growth curve of microbial communities;
S5: determining an optimal subculturing timing for the microorganisms according to the growth curve, and at the optimal subculturing timing, transferring the microbial liquid from a previous-level reaction kettle to a next-level reaction kettle under pressure-retaining conditions; and
S6: repeating steps S3 to S5 to perform long-period enrichment culture and interactive subculturing of the deep-sea microorganism samples in the microorganism enrichment system.
